(19) United States
(12) Patent Application Publication
(10) Pub. No.: US 2025/0352310 A1
(43) Pub. Date: Nov. 20, 2025

(54) METHOD AND SYSTEM FOR DECOUPLING DENTAL ARCHES IN DENTAL SCAN DATA

(71) Applicant: Milan Madhavji, Mississauga (CA)

(72) Inventor: Milan Madhavji, Mississauga (CA)

(21) Appl. No.: 19/209,372

(22) Filed: May 15, 2025

Related U.S. Application Data
(60) Provisional application No. 63/648,993, filed on May 17, 2024.

(51) Int. Cl. A61C 9/00; A61C 7/00; G06T 7/12; G06T 19/00; G16H 30/20

(52) U.S. Cl. CPC A61C 9/0046; A61C 7/002; G06T 7/12; G06T 19/00; G16H 30/20; G06T 2200/04; G06T 2207/20212; G06T 2207/30036; G06T 2210/41

(57) ABSTRACT

A method comprises acquiring a first volumetric image data set representing dentition of a patient, the first volumetric image data set including a modeled upper arch and a modeled lower arch, segmenting the modeled upper arch and the modeled lower arch in the first volumetric image data set, for each of the modeled upper arch and the modeled lower arch, masking a one of the modeled upper arch and the modeled lower arch to obtain decoupled volumetric image data including an other one of the modeled upper arch and the modeled lower arch and, combining the decoupled volumetric image data including the modeled upper arch and the decoupled volumetric image data including the modeled lower arch into a second volumetric image data set representing dentition of the patient and having the modeled upper arch and the modeled lower arch decoupled from one another.

### CROSS-REFERENCE TO RELATED APPLICATIONS

This application claims the benefit of provisional U.S. Patent Application No. 63/648,993 filed May 17, 2024, the contents of which are incorporated herein by reference.

### TECHNICAL FIELD

The present invention relates to a method and system for decoupling dental arches in dental scan data. More specifically, the present invention relates to a method and system for digitally decoupling image data representing dental arches in a volumetric model of dentition of a patient.

### BACKGROUND

Three-dimensional (3D) scanning or volumetric scanning is a technique used to capture detailed three-dimensional image information about objects and their internal structures. In dental radiology, volumetric scanning primarily revolves around the use of Cone Beam Computed Tomography (CBCT). CBCT is an imaging technique that provides three-dimensional (3D) images of the teeth, oral and maxillofacial region. These scans provide valuable information for dental professionals to diagnose and treat dental conditions.

However, 3D scanning, like other forms of radiographic imaging, can face challenges. One common issue is the overlapping of the dental arches or teeth in the 3D scans. A typical human mouth has upper and lower dental arches each with its own row of teeth; one for the upper arch and one for the lower arch. The rows of teeth will have areas of overlap with each other due to interdigitating surfaces when the mouth is closed and the rows of teeth are in contact with each other. Also, teeth which come into contact with each other when the mouth is closed have overlapping topographies. Image data is generally captured as a unitary volume of pixels or voxels and it is not until a segmentation operation is performed that individual components can be isolated or decoupled from one another and then labeled as distinct items. Due to limitations in x-ray radiation detection and image processing post-scanning operation, there is some “fuzziness” as to the specific boundary between the image data representing the teeth of the upper arch and the teeth of the lower arch in the initial set of generated images, even when using very sophisticated x-ray scanning systems. In the resultant images, teeth may appear overlapped due to the specific positioning of the patient or the inherent limitations in how the x-rays penetrate and image the different densities within the oral cavity. This overlap can obscure critical details of the teeth and jaw structure, making it difficult for dental professionals to accurately diagnose or plan treatments. Information such as the alignment of biting surfaces and range of overbite or underbite are difficult to visualize when image data boundaries are not clear. This overlap cannot be addressed by simply cropping out a section of the overlapping arches as it would result in potential data loss and inaccuracies. For instance, cropping out one of the arches can result in the biting surfaces of the teeth being smoothed over or omitted entirely, especially when the boundaries between teeth are inaccurately defined.

To avoid overlapping upper and lower arch image data, separate scanning operations and data captures for the upper arch and the lower arch may be conducted. These scans can be done at separate times, with the patient taking the first scan in a first appointment and then returning for taking the second or additional scans. Alternatively, the scanning operation may be taken with the patient's mouth in an open position to separate the upper and lower arches more distinctly. This method helps to eliminate the overlap seen in a closed mouth position, allowing for clearer visibility of individual teeth and their relation to each other and the jawbone. However, taking additional scans not only causes inconvenience for the patient but also exposes them to additional radiation, potentially increasing risk of other illnesses. Additional imaging also increases the overall cost of diagnosis and treatment for the patient.

Therefore, there is a need for a more efficient method to address the problem of overlapping teeth between upper and lower arch image data in 3D scans.

### BRIEF SUMMARY

The present invention relates to a method and system for decoupling dental arches in dental scan data. More specifically, the present invention relates to a method and system for digitally decoupling image data representing dental arches in a volumetric model of dentition of a patient.

The present invention provides a system and method for digitally masking or removing one of the arches and/or digitally decoupling or separating the upper and lower arches thereby reducing or eliminating the need for additional X-ray scans and reducing the patient's exposure to radiation. The system and method provide further advantage in that the upper and lower arches, once decoupled, are capable of relative movement therebetween. Accordingly, the present invention provides for dynamic adjustment of bite in 3D dental scans, which is advantageous in addressing the limitations posed by overlapping teeth in dental scans. Additionally, this invention provides a more accurate and efficient way to analyze 3D scans, thereby improving the overall quality of dental care. The method may further include exporting the second volumetric image data set to a Digital Imaging and Communications in Medicine (DICOM) file format.

In one aspect, there is provided a method including acquiring a first volumetric image data set representing dentition of a patient, the first volumetric image data set including a modeled upper arch and a modeled lower arch, segmenting the modeled upper arch and the modeled lower arch in the first volumetric image data set, for each of the modeled upper arch and the modeled lower arch, masking a one of the modeled upper arch and the modeled lower arch to obtain decoupled volumetric image data including an other one of the modeled upper arch and the modeled lower arch, and, combining the decoupled volumetric image data including the modeled upper arch and the decoupled volumetric image data including the modeled lower arch into a second volumetric image data set representing dentition of the patient and having the modeled upper arch and the modeled lower arch decoupled from one another.

The method may further include modifying the second volumetric image data set to include at least one reference point relative to which one of the modeled upper arch and the modeled lower arch is repositioned relative to the other one of the modeled upper arch and the modeled lower arch. The one of the modeled upper arch and the modeled lower arch may be selectively repositioned relative to the other one of the modeled upper arch and the modeled lower arch.

The reference point may be a pivot point about which one of the modeled upper arch and the modeled lower arch is rotated for repositioning the one of the modeled upper arch and the modeled lower arch relative to the other one of the modeled upper arch and the modeled lower arch. The one of the modeled upper arch and the modeled lower arch may be rotatable about the pivot point between an open bite position wherein the modeled upper arch and the modeled lower arch are in contact with one another and a closed bite position wherein the modeled upper arch and the modeled lower arch are spaced apart.

Masking may further include providing a horizontal boundary one of above and below an occlusal plane between the modeled upper arch and the modeled lower arch and, masking one of the modeled upper arch and the modeled lower arch that extends from the horizontal boundary and away from the other one of the modeled upper arch and the modeled lower arch. In one aspect, masking further includes masking portions of the one of the modeled upper arch and the modeled lower arch that extend beyond the horizontal boundary toward the other one of the modeled upper arch and the modeled lower arch.

In one aspect, a system includes a capture module configured to acquire a first volumetric image data set representing dentition of a patient, the first volumetric image data set including a modeled upper arch and a modeled lower arch. A segmentation module is configured to segment the modeled upper arch and the modeled lower arch in the first volumetric image data set. A masking module is configured to, for each of the modeled upper arch and the modeled lower arch, mask a one of the modeled upper arch and the modeled lower arch to obtain decoupled volumetric image data including an other one of the modeled upper arch and the modeled lower arch. A modeling module is configured to combine the decoupled volumetric image data including the modeled upper arch and the decoupled volumetric image data including the modeled lower arch into a second volumetric image data set representing dentition of the patient and having the modeled upper arch and the modeled lower arch decoupled from one another. The system may further include an export module configured to export the second volumetric image data set to a Digital Imaging and Communications in Medicine (DICOM) file format.

The system may further include a positioning module configured to modify the second volumetric image data set to include at least one reference point relative to which one of the modeled upper arch and the modeled lower arch is repositioned relative to the other one of the modeled upper arch and the modeled lower arch. The one of the modeled upper arch and the modeled lower arch may be selectively repositioned relative to the other one of the modeled upper arch and the modeled lower arch. In one aspect, the reference point is a pivot point about which one of the modeled upper arch and the modeled lower arch is rotatable for repositioning the one of the modeled upper arch and the modeled lower arch relative to the other one of the modeled upper arch and the modeled lower arch. The one of the modeled upper arch and the modeled lower arch may be rotatable about the pivot point between an open bite position wherein the modeled upper arch and the modeled lower arch are in contact with one another and a closed bite position wherein the modeled upper arch and the modeled lower arch are spaced apart.

The masking module may be further configured to provide a horizontal boundary one of above and below an occlusal plane between the modeled upper arch and the modeled lower arch and, mask one of the modeled upper arch and the modeled lower arch that extends from the horizontal boundary and away from the other one of the modeled upper arch and the modeled lower arch. The masking module may be further configured to mask portions of the one of the modeled upper arch and the modeled lower arch that extend beyond the horizontal boundary toward the other one of the modeled upper arch and the modeled lower arch.

In one aspect, a non-transitory computer-readable medium having instructions stored thereon which, when executed on a processor, performs the steps of acquiring a first volumetric image data set representing dentition of a patient, the first volumetric image data set including a modeled upper arch and a modeled lower arch, segmenting the modeled upper arch and the modeled lower arch in the first volumetric image data set for each of the modeled upper arch and the modeled lower arch, masking a one of the modeled upper arch and the modeled lower arch to obtain decoupled volumetric image data including an other one of the modeled upper arch and the modeled lower arch and, combining the decoupled volumetric image data including the modeled upper arch and the decoupled volumetric image data including the modeled lower arch into a second volumetric image data set representing dentition of the patient and having the modeled upper arch and the modeled lower arch decoupled from one another.

The non-transitory computer-readable medium may further have instructions stored thereon for performing the step of modifying the second volumetric image data set to include at least one reference point relative to which one of the modeled upper arch and the modeled lower arch is repositioned relative to the other one of the modeled upper arch and the modeled lower arch. The one of the modeled upper arch and the modeled lower arch is selectively repositioned relative to the other one of the modeled upper arch and the modeled lower arch. The reference point may be a pivot point about which one of the modeled upper arch and the modeled lower arch is rotated for repositioning the one of the modeled upper arch and the modeled lower arch relative to the other one of the modeled upper arch and the modeled lower arch.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system for decoupling dental arches in dental scan data in accordance with one aspect;

FIG. 2 illustrates a method for decoupling dental arches in dental scan data in accordance with one aspect;

FIG. 3 illustrates an image processing module for decoupling dental arches in dental scan data in accordance with one aspect;

FIG. 4 illustrates image data representing dentition of a patient in accordance with one aspect;

FIG. 5 illustrates segmented image data representing dentition of a patient in accordance with one aspect;

FIG. 6 illustrates decoupled image data representing dentition of a patient in accordance with one aspect;

FIG. 7 illustrates decoupled image data representing dentition of a patient in accordance with one aspect;

FIG. 8 illustrates decoupled image data representing dentition of a patient in accordance with one aspect;

FIG. 9 illustrates decoupled image data representing dentition of a patient in accordance with one aspect; and,

FIG. 10 illustrates second volumetric image data set representing dentition of a patient in accordance with one aspect.

### DETAILED DESCRIPTION

The present invention relates to a method and system for decoupling dental arches in dental scan data. More specifically, the present invention relates to a method and system for digitally decoupling image data representing dental arches in a volumetric model of dentition of a patient.

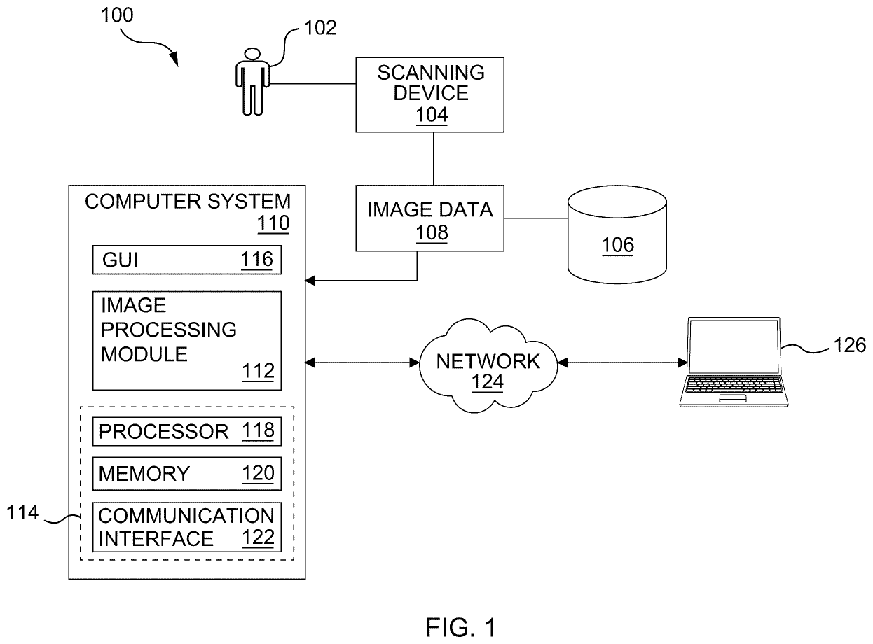

FIG. 1 illustrates a system 100 for digitally decoupling image data representing upper and lower dental arches in a volumetric model of dentition of a patient, in accordance with one aspect.

System 100 includes computer system 110 for analyzing image data 108 representing dentition of a patient 102. Image data 108 is acquired using a scanning device 104 which may then be provided directly to computer system 110 or which may be retrieved by computer system 110 from data storage 106. Scanning device 104 may be any suitable scanning device such as intraoral scanners, cone beam computed tomography (CBCT) scanners, x-ray machines, and the like.

Image data 108 is preferably three-dimensional image data 108 and in a format of or capable of being converted into a volumetric model including volumetric representations of the various dental structures of the patient 102, including the upper and lower jawbones, and surrounding tissues of the patient 102. In the context of a three-dimensional model, such representations may be referred to as “modeled structures”. In one aspect, image data 108 is acquired in Digital Imaging and Communications in Medicine (DICOM) format. DICOM is a universal standard for the storage, handling, and transmission of medical images and associated information. It ensures compatibility and interoperability among different systems and devices by standardizing the format and including comprehensive metadata such as patient identification, image type, and device information. DICOM files are integral in maintaining the integrity and consistency of medical data as they include both the image and its complete context-information critical to accurate diagnosis and treatment planning.

Computer system 110 includes a controller 114, a graphical user interface (GUI) 116, and an image processing module 112. The controller 114 includes at least one processor 118, a memory 120 configured to store one or more first program instructions and at least one communication interface 122.

The processor 118 may include one or more processing elements, micro-controllers, circuitry, field programmable gate array (FPGA) or other processing system, and resident or external memory for storing data, executable code, and other information accessed or generated by the computer system 110. Therefore, processor 118 may include any microprocessor device configured to execute algorithms or program instructions. In general, the term “processor”, may be broadly defined to encompass any device having one or more processing elements, which execute a set of program instructions from one or more processing elements, which execute a set of program instructions from a non-transitory memory medium, where the set of program instructions is configured to cause the one or more processors to carry out any of the one or more process steps.

The memory 120 may include any storage medium known in the art suitable for storing the set of program instructions executable by the associated one or more processors. For example, memory 120 may include a non-transitory memory medium. Memory 120 may include but is not limited to, a read-only memory (ROM), a random access memory (RAM), a magnetic or optical memory device (e.g., disk), a magnetic tape, a solid state drive, flash memory (e.g., a secure digital (SD) memory card, a mini-SD memory card, and/or a micro-SD memory card), universal serial bus (USB) memory devices, and the like. The memory 120 may be housed in a common controller housing with the one or more processors. Alternatively or in addition, the memory 120 may be located remotely with respect to the spatial location of the processors and/or the controller 114 may access a remote memory (e.g., server), accessible through a network (e.g., internet, intranet, and the like).

The controller 114 may be configured to perform one or more process steps, as defined by the one or more sets of program instructions. The one or more process steps may be performed iteratively, concurrently and/or sequentially. The one or more sets of program instructions may be configured to operate via a control algorithm, a neural network (e.g., with states represented as nodes and hidden nodes and transitioning between them until an output is reached via branch metrics), a kernel-based classification method, a Support Vector Machine (SVM) approach, canonical-correlation analysis (CCA), factor analysis, flexible discriminant analysis (FDA), principal component analysis (PCA), multidimensional scaling (MDS), principal component regression (PCR), projection pursuit, data mining, prediction-making, exploratory data analysis, supervised learning analysis, Boolean logic (e.g., resulting in an output of a complete truth or complete false value), fuzzy logic (e.g., resulting in an output of one or more partial truth values instead of a complete truth or complete false value), or the like. For example, in the case of a control algorithm, the one or more sets of program instructions may be configured to operate via proportional control, feedback control, feedforward control, integral control, proportional-derivative (PD) control, proportional-integral-derivative (PID) control, or the like.

The communication interface 122 may be operatively configured to communicate with one or more components of the computer system 110 and/or controller 114. For example, communication interface 122 may also be coupled (e.g., physically, electronically, and/or communicatively) with the at least one processor 118 to facilitate data transfer between components of the computer system 110, other components of system 100 and processor 118. For instance, the communication interface 122 may be configured to retrieve data from the at least one processor 118, or other devices, transmit data for storage in the memory 120, retrieve data from storage in the memory 120, or the like. By way of another example, controller 114 may be configured to receive and/or acquire data or information from other systems or tools by a transmission medium that may include wireline and/or wireless portions. By way of another example, controller 114 may be configured to transmit data or information (e.g., the output of one or more procedures of the inventive aspects disclosed herein) to one or more systems or tools by a transmission medium that may include wireline and/or wireless portions (e.g., a transmitter, receiver, transceiver, physical connection interface or any combination thereof). In this regard, the transmission medium may serve as a data link between the controller 114 and the other components of the computer system 110 and system 100. In addition, controller 114 may be configured to send data to external systems via a transmission medium (e.g., network connection).

In general, the word “module” as used herein, refers to a collection of hardware components and/or software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software may be stored, partially or fully, on a memory device of the executing computing device, for execution by the computing device. Software instructions may be embedded in firmware, such as an EPROM. It will be further appreciated that hardware devices (such as processors and CPUs) may be comprised of connected logic units, such as gates and flip-flops, and/or may be comprised of programmable units, such as programmable gate arrays or processors. The modules or computing device functionality described herein are preferably implemented as software modules but may be represented in hardware devices. Generally, the modules described herein refer to hardware or software modules that may be combined with other modules or divided into sub-modules despite their physical organization or storage.

Embedded within or accessible to the computer system 110 is image processing module 112. “Image processing module” refers to one or more computer components, which may include hardware or software, which are designed to collect, create, edit, process, analyze, and display image data. Such components may be local to the image processing module or external and in data exchange communication therewith. Image processing module is preferably medical image processing module configured to manage and process images obtained from various diagnostic tools such as X-rays, CT scans, MRIs, ultrasound, and other imaging modalities. Image processing module 112 can handle various image formats from simple photographs to complex graphics and medical scans. Image processing module 112 may exist in various forms, such as being embedded on a hard drive of computer system 110, stored on a server in data communication with computer system 110 or is accessible as a third-party software that can be used as a service by computer system 110. In another aspect, image processing module 112 may include one or more machine learning models or an “artificial intelligence” system capable of performing automated image analysis, accessible by computer system 110. Image processing module 112 is configured to receive as input image data 108 acquired from the scanning device 104 or images stored in data storage 106 or elsewhere that is accessible by image processing module 112. Image processing module 112 may acquire the image data 108 automatically as a function of system 100 or may be instructed to acquire image data 108 by user input via graphical user interface 116, with graphical user interface 116 being in data exchange communication with image processing module 112. Once image data 108 is acquired by computer system 110 and is accessible to image processing module 112, image processing module 112 is configured to delineate various modeled structures of the patient's dentition, selectively decouple and/or mask various structures, and selectively enable relative movement between modeled jaw or arch structures and to output a second image data set which, in some aspects, may be a digitally altered, reconstructed or modified image data set. Image processing module 112 can enhance the visibility of anatomical structures, improve image quality by digitally decoupling or separating the modeled upper and lower jaws to eliminate overlapping of modeled teeth. Use of imaging software can lead to a more accurate and efficient way to analyze 3D or CBCT scan data, improving the overall quality of dental care.

The computer system 110 is in data exchange communication with a user device 126 via network 124. The network 124 may comprise any suitable network or networks, including a local area network (LAN), wide area network (WAN), Internet, or combination thereof. For example, the network 124 may include a wireless cellular service (e.g., 4G). Generally, the network 124 enables bidirectional communication between the computer system 110 and the user device 126. In some aspects, network 124 may comprise a cellular base station, such as cell tower(s), communicating to the one or more components of the system 100 via wired/wireless communications based on any one or more of various mobile phone standards, including NMT, GSM, CDMA, UMMTS, LTE, 5G, or the like. Additionally or alternatively, network 124 may comprise one or more routers, wireless switches, or other such wireless connection points communicating to the components of the computer system 110 via wireless communications based on any one or more of various wireless standards, including by non-limiting example, IEEE 802.11a/b/c/g (WIFI), the BLUETOOTH standard, or the like.

User device 126 may be any suitable device, for example, a laptop, a computer, a smart phone, a tablet, and the like. User device 126 is operable by a healthcare practitioner to access the digitally adjusted images of the patient 102 created using the image processing module 112 of the computer system 110.

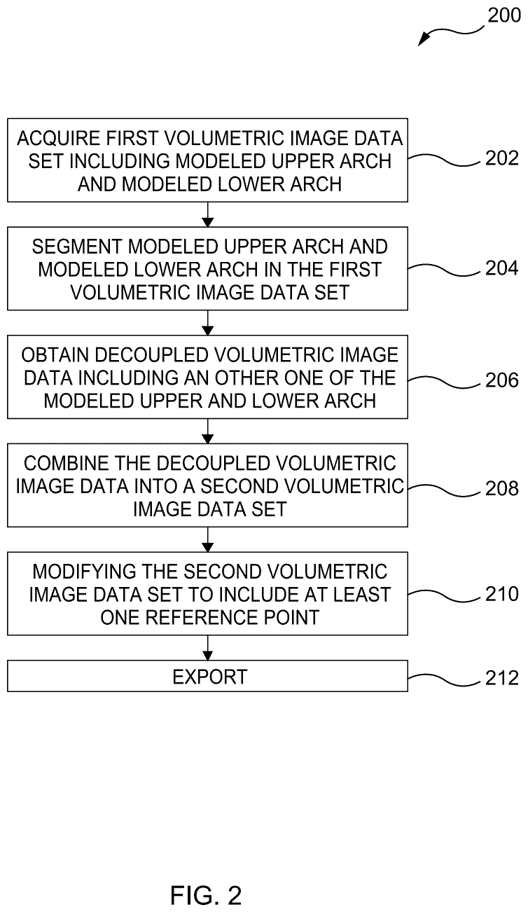

In FIG. 2, there is shown a method 200 for decoupling image data representing dental arches in a volumetric model of dentition of a patient, according to one aspect. 3D scans in dental applications may be taken in a closed bite position or an open bite position, depending on patient and/or imaging technician preference. A closed bite dental scan may be favored because it accurately reflects the natural occlusion, showing how the upper and lower teeth meet, which provides a realistic assessment of the patient's bite alignment and jaw alignment. A closed bite dental scan also helps stabilize the jaw during the scan, minimizing movement and enhancing image clarity. While a closed bite dental scan is commonly used for diagnosing and planning treatments, it may provide limited visibility in some areas, particularly the occlusal surfaces and interproximal spaces. Additionally, the overlay of dental surfaces in a closed bite scan can lead to overlaps in the imaging data, making it challenging to interpret the condition of underlying structures accurately. Further, certain specific clinical needs might necessitate scans in an open bite position, depending on the focus of the diagnostic examination. The method 200 of FIG. 2 provides a technique for altering or adjusting the dental arches in a 3D dental scan taken in one position to generate a modified image data set in a different position without losing image data or clarity.

The method 200 includes, as shown at block 202, acquiring a first volumetric image data set representing dentition of a patient. The first volumetric image data set is the image data 108 of FIG. 1, in one aspect. The first volumetric image data set includes a modeled upper arch 402 and a modeled lower arch 404 representing corresponding upper and lower dental arches of the patient's dentition, respectively.

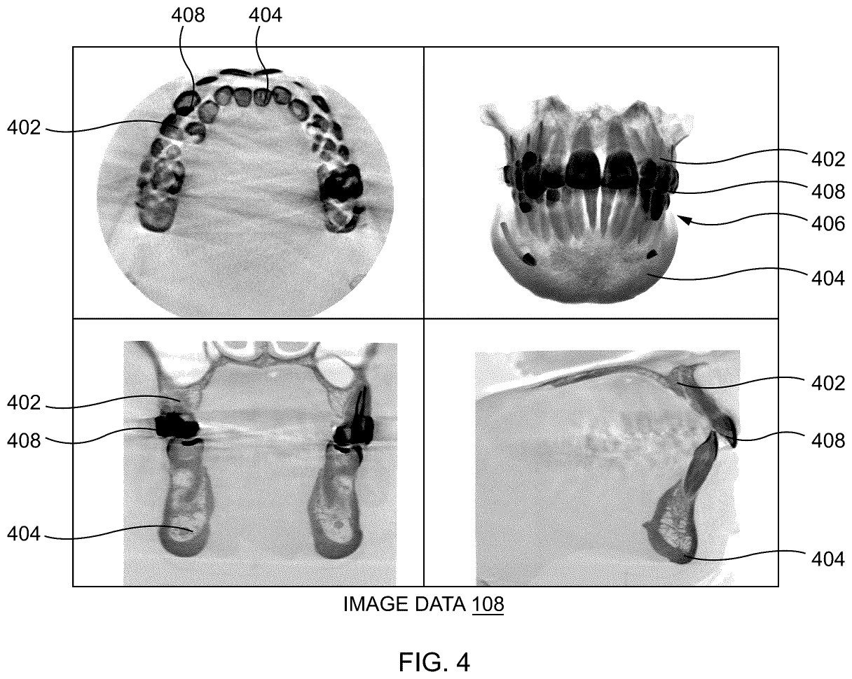

An example CBCT scan data of a patient's dentition is shown in FIG. 4. As illustrated in FIG. 4, the CBCT scan data in the closed bite position may include some overlap between the modeled maxillary and mandibular teeth. Areas of overlap may include, for example, interdigitating surfaces of modeled teeth or molars between the modeled upper arch and modeled lower arch. In one aspect, the segmentation step of block 204 includes segmenting individual structures such as modeled teeth of the modeled upper arch 402 and modeled lower arch 404 in the first volumetric image data set.

At block 204, the modeled upper arch 402 and the modeled lower arch 404 are segmented in the first volumetric image data set. Segmentation refers to the process of isolating and distinguishing distinct structures and sub-structures in the first volumetric image data set and can be carried out using known techniques. Various specialized software tools are available that can perform dental segmentation, often utilizing artificial intelligence (AI) to enhance accuracy and efficiency. In some aspects, once the segmentation is complete, the data, which originally is in voxel format, may be used to generate a mesh. This segmented mesh is a collection of vertices, edges, and faces that approximate the shape of each original structure or teeth in 3D space.

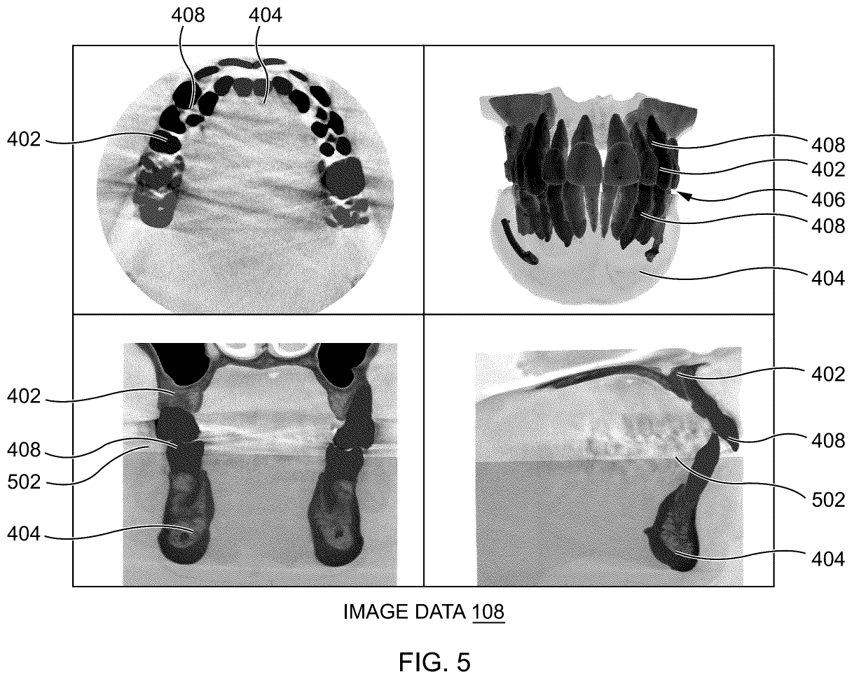

FIG. 5 shows the image data 108, or first volumetric image data set, representing patient dentition of FIG. 4, with individual modeled structures, including those of the modeled upper and lower arches 402, 404 segmented using suitable segmentation techniques. Segmentation refers to the technique of delineating and separating overlapping anatomical regions within an image data set.

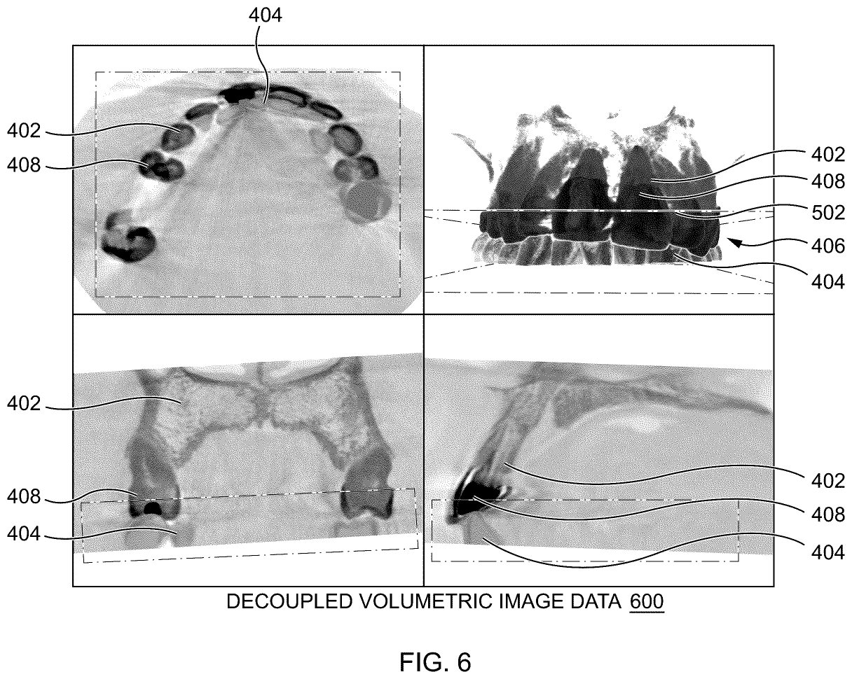

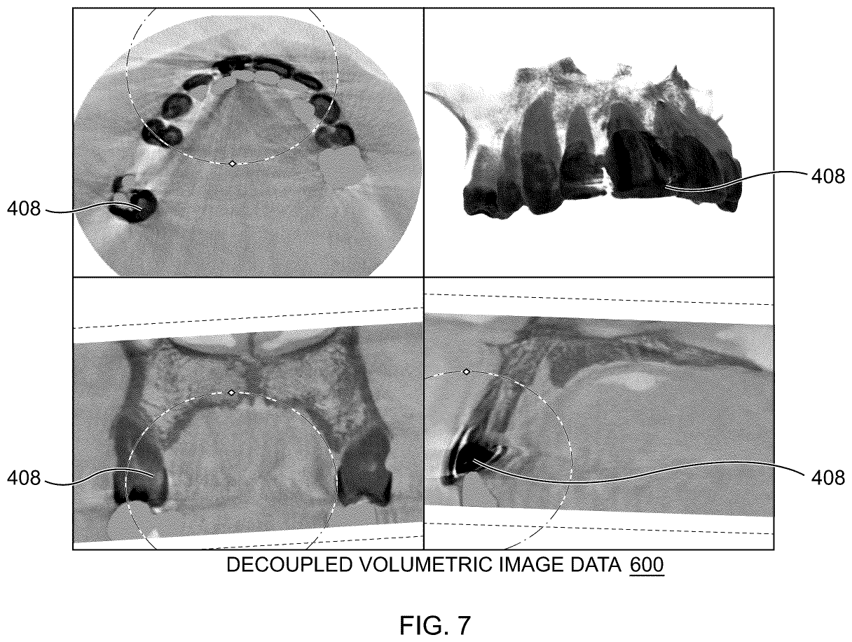

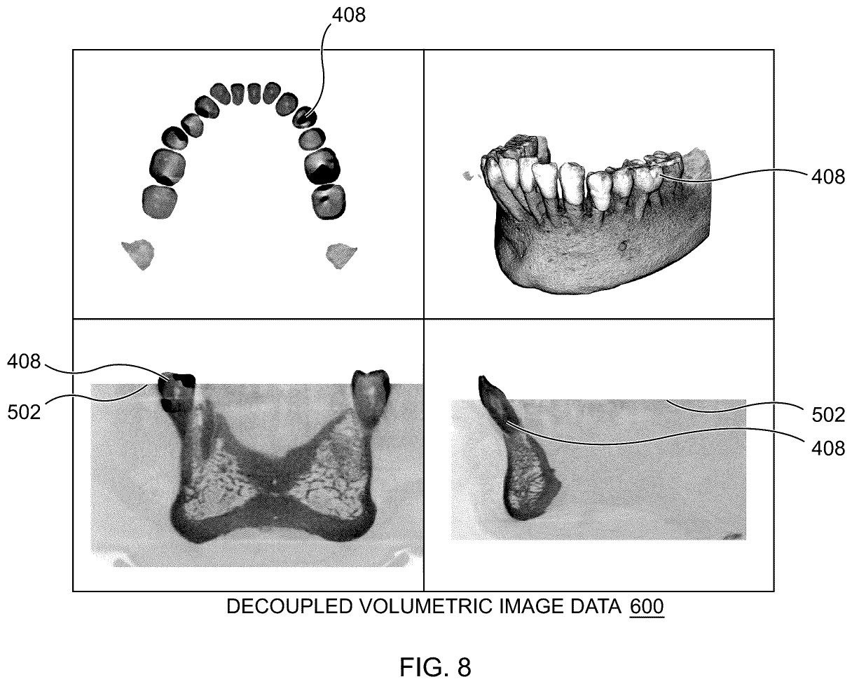

At block 206, the method 200 includes, for each of the modeled upper arch 402 and the modeled lower arch 404, masking a one of the modeled upper arch 402 and the modeled lower arch 404 to obtain decoupled volumetric image data 600 (FIG. 6) including an other one of the modeled upper arch 402 and the modeled lower arch 404. “Masking” may include covering the segmented meshes of the modeled upper arch 402 and/or the modeled lower arch 404, altering their respective properties to make them non-visible or removing them from the first volumetric image data set to obtain isolated scan data for the other one of the other one of the modeled upper arch 402 and the modeled lower arch 404, as shown in FIG. 7, wherein the modeled lower arch 404 is masked and FIG. 8, wherein the modeled upper arch 402 is masked.

Masking of all structures of the modeled upper arch 402 or the modeled lower arch 404 along the occlusal plane 406 (FIG. 4) can be challenging due to the large number of interdigitating surfaces between the rows of modeled teeth 408 of the modeled upper and lower arches 402, 404. Also, x-ray imaging is imperfect and, even at high-resolutions, the first volumetric image data set may include unclear or “fuzzy” boundaries between contacting structures which can sometimes lead to segmentation errors. To facilitate masking of components of the first volumetric image data set, particularly with respect to those along the occlusal plane 406, the method 200 may include drawing of a horizontal boundary 502, such as a line or plane, above or below the occlusal plane 406, where the modeled upper arch 402 and the modeled lower arch 404 overlap. Once the horizontal boundary 502 is drawn, as shown in FIG. 5, a portion of the first volumetric image data set above or below the horizontal boundary 502 may be masked, leaving one of the upper and lower arch completely visible along with a portion of the other one of the upper and lower arch.

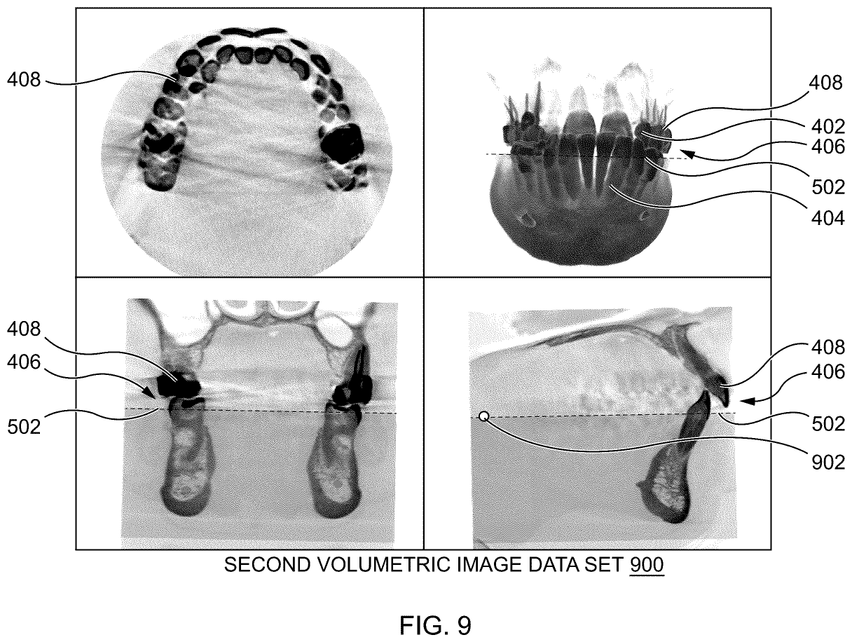

For example, in the aspect shown in FIG. 6 and FIG. 9, the horizontal boundary 502 is drawn below the occlusal plane 406 and a portion of the modeled lower arch 404 below the horizontal boundary 502 is masked. This separates a large portion of the modeled lower arch 404 from the modeled upper arch 402. The portions of the modeled lower arch 404 that remain visible and which overlap with the modeled upper arch 402 are mainly portions of segmented modeled teeth 408 which may be further masked by identifying and separating the cusp tips and grooves of the individual segmented teeth.

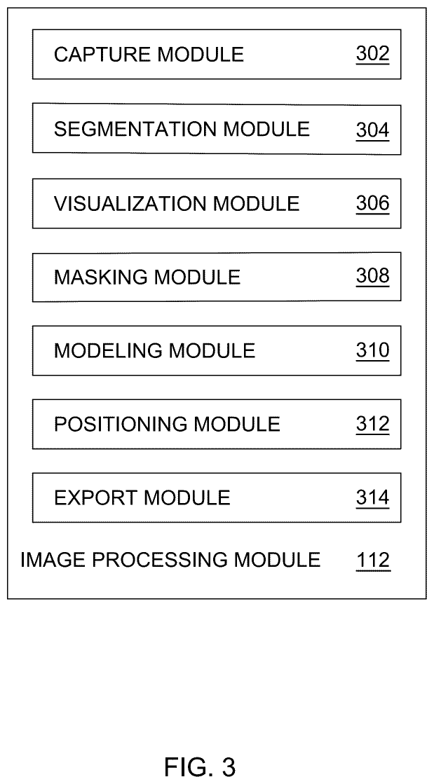

At block 208, the decoupled volumetric image data 600 (FIG. 6) including the modeled upper arch 402 and the decoupled volumetric image data 600 including the modeled lower arch 404 are combined into a second volumetric image data set 900 (FIG. 9) representing dentition of the patient and having the modeled upper arch 402 and the modeled lower arch 404 decoupled from one another. Such a combination may be provided using a modeling module 310, an example of which is shown in FIG. 3.

At block 210, the method 200 may proceed to modifying the second volumetric image data set 900 to include at least one reference point 902 (FIG. 9). One of the modeled upper arch 402 and the modeled lower arch 404 may be repositioned relative to the other one of the modeled upper arch 402 and the modeled lower arch 404 relative the reference point 902. In one aspect, the reference point 902 is a pivot point about which one of the modeled upper arch 402 and the modeled lower arch 404 is rotated for repositioning the one of the one of the modeled upper arch 402 and the modeled lower arch 404 relative to the other one of the modeled upper arch 402 and the modeled lower arch 404.

This aspect has advantage in that dynamically adjusting a position of the upper arch with respect to the lower arch using the pivot point to simulate or create a new data set or image of the patient's dentition that shows the dental arches in a different position than the original scan. The one of the modeled upper arch and the modeled lower arch may be rotated about the pivot point between an open bite position wherein the modeled upper arch and the modeled lower arch are in contact with one another and a closed bite position wherein the modeled upper arch and the modeled lower arch are spaced apart. Thereby, the open and closed bite positions of the patient may be digitally modified, corrected and/or planned. This may save substantial amounts of time during treatment as the clinically appropriate bite position may be determined during the planning phase, thereby reducing or eliminating corrective adjustments during treatment. For example, a new data set showing the dental arches in a different position may be used to adjust the position of individual teeth and the jaw alignment to simulate the correct bite. This helps in planning orthodontic treatment or surgery.

The method 200 may further include exporting the digitally created scan to a DICOM format, as shown at block 212. DICOM is the standard format for handling, storing, and transmitting information in medical imaging. Exporting the digitally simulated scan into DICOM format allows the data to be easily shared and accessed across different medical imaging systems and platforms used by various healthcare professionals. The exported DICOM files of segmented teeth can be utilized in various dental software tools for further analysis, treatment planning, and even for the creation of orthodontic appliances or surgical guides.

FIG. 3 illustrates an image processing module 112 for decoupling image data representing dental arches 402, modeled lower arch 404 in a volumetric model of dentition of a patient, in accordance with one aspect.

Image processing module 112 includes a capture module 302 configured to acquire the first volumetric image data set representing dentition of the patient. As described with respect to FIG. 1 and FIG. 2, the first volumetric image data set including a modeled upper arch and a modeled lower arch.

Image processing module 112 includes segmentation module 304 configured to segment the modeled upper arch 402 and the modeled lower arch 404 in the first volumetric image data set received from capture module 302. Segmentation module 304 utilizes advanced algorithms to automatically or semi-automatically distinguish and isolate different anatomical structures within the dental scans, such as teeth, bones, nerves, and soft tissues. By accurately segmenting these structures, segmentation module 304 provides dentists and oral surgeons with precise, detailed visualizations that aid in diagnosis, treatment planning, and surgical simulation. The segmentation module 304 may include a user-friendly interface that allows dental professionals to refine and adjust the segmentation to accommodate individual anatomical variations, thereby enhancing the accuracy and effectiveness of dental treatments. Additionally, the integration of machine learning and artificial intelligence in segmentation can further improve the speed and accuracy of the segmentation process.

Image processing module 112 includes visualization module 306, which provides tools for customizing the visual representation of the first volumetric image data set segmented by the segmentation module 304. Visualization module 306 may include options for visual enhancement, such as adjusting transparency, color intensity, and applying different color maps to various tissue for more detailed analysis. Visualization module 306 enhances the visualization of the segmented structures. Visualization module 306 may automatically assign different colors for different anatomical structures like teeth, bones, nerves, and soft tissues, segmented by the segmentation module 304. This color differentiation helps in easily distinguishing these structures visually, aiding in better assessment and planning. For example, different types of teeth may be highlighted in different colors within the segmentation view as illustrated in FIG. 5. Visualization module 306 may be an embedded or integrated component of the segmentation module 304 or may be separate from the segmentation module 304 and in data exchange communication therewith.

In one aspect, visualization module 306 further includes a range of tools and/or algorithms that allow dental professionals to adjust image parameters such as brightness, contrast, and sharpness, as well as apply advanced processing techniques like noise reduction, edge enhancement, and geometric transformations like rotation and scaling. Such tools may also include specialized functions such as panoramic reconstruction and the ability to filter specific frequencies to highlight particular structures, such as soft tissues or dense bony areas.

Image processing module 112 further includes a masking module 308 configured to, for each of the modeled upper arch 402 and the modeled lower arch 404, mask a one of the modeled upper arch 402 and the modeled lower arch 404 to obtain decoupled volumetric image data 600 including an other one of the modeled upper arch 402 and the modeled lower arch 404. In one aspect, the masking module 308 receives the first volumetric image data set segmented by the segmentation module 304 and as colored by the visualization module 306, where visualization module 306 has modified the first volumetric image data set. In one aspect, the masking module 308 masks at least one of the modeled upper arch 402 and the modeled lower arch 404. This may be accomplished using one or more tools of the masking module 308 for selectively masking or digitally removing each of the modeled arches in the first volumetric image data set.

Image processing module 112 further includes a modeling module 310 configured to combine the decoupled volumetric image data 600 including the modeled upper arch 402 and the decoupled volumetric image data 600 including the modeled lower arch 404 into a second volumetric image data set 900 representing dentition of the patient and having the modeled upper arch 402 and the modeled lower arch 404 decoupled from one another.

Image processing module 112 further includes positioning module 312 which is configured to modify the second volumetric image data set 900 to include at least one reference point 902 relative to which one of the modeled upper arch 402 and the modeled lower arch 404 is repositioned relative to the other one of the modeled upper arch 402 and the modeled lower arch 404. In one aspect, the reference point 902 is a pivot point about which one of the modeled upper arch 402 and the modeled lower arch 404 is rotatable for repositioning the one of the modeled upper arch 402 and the modeled lower arch 404 relative to the other one of the modeled upper arch 402 and the modeled lower arch 404.

Positioning module 312 may further include inputs for repositioning the modeled upper arch 402 and modeled lower arch 404 about the reference point 902 once the reference point 902 is established. Such inputs may include user-input controls such as “drag and drop” controls or directional inputs which may be interacted with by a user to reposition the upper and/or lower arch. Such inputs facilitate dynamic adjustment of a position of the upper arch with respect to the lower arch using the reference point or pivot point so that the patient's arches and dentition may be viewed in a position that is different from the positions shown in the first volumetric image data set. This is advantageous, for example, in that the patient's dentition may be adjusted within the volumetric image data set to simulate a correct bite and to identify the dental adjustments needed to achieve that correct bite. This helps in planning orthodontic treatment or surgery.

Image processing module 112 further includes an export module 314 configured to export the second volumetric image data set 900 to a suitable file format such as Digital Imaging and Communications in Medicine (DICOM) file format. Thereby, exchange of the second volumetric image data set 900 between systems may be facilitated.

FIG. 4 illustrates a quadrant view of a volumetric model of a patient's dentition and particularly illustrating spatial relationships between the modeled upper and lower dental arches in a closed-bite position. The model comprises three-dimensional digital representations of the teeth of the modeled maxillary (upper) and mandibular (lower) arches in a simulated occlusal position, with opposing teeth shown in overlapping contact to reflect interdigitation of occlusal surfaces. FIG. 4 is divided into four quadrants, as would be presented to a user of diagnostic software common to dental practitioners and radiologists. Each quadrant provides a distinct sectional or perspective view of the modeled dentition, intended to highlight various aspects of occlusion, inter-arch relationships, and diagnostic features relevant to dental assessment and treatment planning. Although FIG. 4 illustrates the volumetric model in a closed-bite position, it should be understood that the image data and volumetric model may be provided in an open-bite position, in another aspect.

In the first quadrant (top-left) of FIG. 4, there is shown a frontal perspective view of the volumetric model, depicting the modeled upper arch 402 and modeled lower arch 404 in their natural occlusal arrangement when in a closed-bite position. The anterior modeled teeth 408, including the central and lateral incisors and canines, are shown in overlapping contact. This view emphasizes the alignment, spacing, and interproximal contacts of the anterior dentition, providing insight into midline alignment, overbite, and overjet conditions.

In the second quadrant (top-right) of FIG. 4, there is shown a horizontal cross-sectional view taken along a plane intersecting the occlusal or bite plane. The section transects modeled teeth of both the modeled upper arch 402 and modeled lower arch 404, capturing the occlusal surfaces of opposing teeth. The view facilitates visualization of the contact areas and occlusal morphology, including cusp-fossa relationships and interdigitation patterns. This view is useful in assessing occlusal fit and identifying functional contacts or interferences.

In the third quadrant (bottom-left) of FIG. 4, there is shown a cross-sectional view taken along a vertical plane intersecting the left and right molar regions of both modeled arches 402, 404. The section reveals the occlusal and buccal-lingual relationships of the molars. This view provides information regarding inter-arch alignment and occlusal surface overlap in the posterior dentition, which is advantageous for evaluating occlusal balance and arch coordination.

In the fourth quadrant (bottom-right) of FIG. 4, there is shown a sagittal sectional view through the anterior region, specifically intersecting the central incisor area of both modeled arches. This view illustrates the vertical overlap (overbite) and horizontal overlap (overjet) between the modeled upper and lower incisors. It enables assessment of anterior guidance, incisal edge positioning, and potential discrepancies in anterior tooth angulation.

Collectively, the quadrant views of FIG. 4 offer a comprehensive diagnostic perspective of the patient's modeled dentition, facilitating detailed evaluation of occlusal relationships, inter-arch contacts, and morphological features. The first volumetric image data set and/or volumetric model serves as a useful tool for planning restorative or orthodontic treatments.

FIG. 5 illustrates the same quadrant views and sectional orientations as shown in FIG. 4, but with the individual dental structures of the patient's dentition segmented by segmentation module 304 and preferably labeled and color-coded for enhanced visibility and identification by a practitioner.

Following segmentation, each modeled upper arch 402, modeled lower arch 404, tooth, molar or other relevant anatomical structure (e.g., crowns, roots, interproximal spaces, occlusal surfaces) is a distinct component of the first volumetric image data set or volumetric model and may be provided a distinct visual representation. This enables selective analysis and visualization of specific regions of interest. The segmentation operation may be based on predefined anatomical landmarks, AI-assisted detection, or practitioner-guided modeling or some other suitable method.

In FIG. 5, segmented modeled components are colored or color-coded for ease of distinction between different components, such as tooth types (e.g., incisors, canines, premolars, molars) or distinct modeled structures (e.g., modeled teeth, maxilla, mandible). Modeled structures may be labeled using an appropriate dental notation to facilitate identification and cross-referencing with diagnostic or treatment records. In other aspects, transparency or isolation views may be provided wherein surrounding anatomy is rendered semi-transparent or hidden, allowing focused inspection of specific teeth, root structures, or occlusal surfaces. Occlusal contact mapping may be shown with visual indicators (e.g., color gradients or force vectors) overlaid on contact points to reflect pressure or alignment at the occlusal interface. In another aspect, spacing between adjacent modeled structures may be highlighted to assist in evaluating tightness of contacts, or the presence of gaps. Also, the visualization elements discussed above may be provided in an overlay format, allowing practitioners to toggle between unsegmented and segmented views, or to superimpose anatomical and diagnostic markers.

The segmented model supports enhanced diagnostic interpretation, planning of restorative or orthodontic procedures, and communication with patients or other dental professionals.

FIG. 6 shows the decoupled volumetric image data 600 with components segmented by the segmentation module 304 and colored by the visualization module 306. The horizontal boundary 502 has been positioned below the occlusal plane 406 and the modeled lower arch 404 has been masked by masking module 308, leaving the modeled upper arch 402 and a portion of the modeled lower arch 404 which may be further masked by identifying and separating the cusp tips and grooves of the individual segmented teeth. The original CBCT scan data used to provide the view shown in FIG. 6 was acquired in a “closed bite” position.

FIG. 7 shows the first volumetric image data set with the modeled lower arch 404, including the remaining portions thereof shown in FIG. 6, masked or removed by the masking module 308.

FIG. 8 shows the decoupled volumetric image data 600 with the modeled upper arch 402 and surrounding structures masked or removed by masking module 308. Between the aspects shown in FIG. 7 and FIG. 8, there is provided decoupled volumetric image data 600 representing each of the modeled upper arch 402 602 and the modeled lower arch 404.

The digitally simulated scans of the upper and lower arches 402, 404 retain the clarity and detail of the original scan. For instance, as can be seen from FIG. 7 and FIG. 8, the biting surfaces of modified scans of the upper arch and the lower arch are intact.

The decoupled volumetric image data may be combined by modeling module 310 to provide a second volumetric image data set 900 representing dentition of the patient and having the modeled upper arch and the modeled lower arch decoupled from one another.

FIG. 9 shows the second volumetric image data set 900 of a patient's dentition with the modeled upper arch 402 and the modeled lower arch 404 decoupled from one another. A horizontal boundary 502 is drawn below the plane where the modeled upper arch 402 overlaps with the modeled lower arch 404 for ease of reference. In the aspect shown in FIG. 9, the horizontal boundary 502 separates the two modeled arches.

Using positioning module 312, a reference point 902 and preferably a pivot point, may be positioned along the horizontal boundary 502 at a desired location. In the aspect shown in FIG. 9, the pivot point is located at the approximate position in the image data that would represent the temporomandibular joint (TMJ), which is a pair of joints that connect opposing ends of the mandible to the skull. Once the pivot point is positioned, the modeled upper arch and modeled lower arch may be moved relative to one another, about the pivot point, in a manner which simulates movement of the patient's jaw about the TMJ. Therefore, the creation of the pivot point provides for movement of displacement, particularly hinging movement, of the image data representing the upper arch relative to the image data representing the lower arch in a manner similar to relative movement between the mandible and maxilla about the TMJ. It should be understood that the pivot point may be positioned elsewhere within the image data set should a different outcome be desired. For example, positioning the pivot point differently may be used for locating and testing a corrective treatment to the hinging movement of a patient's jaw.

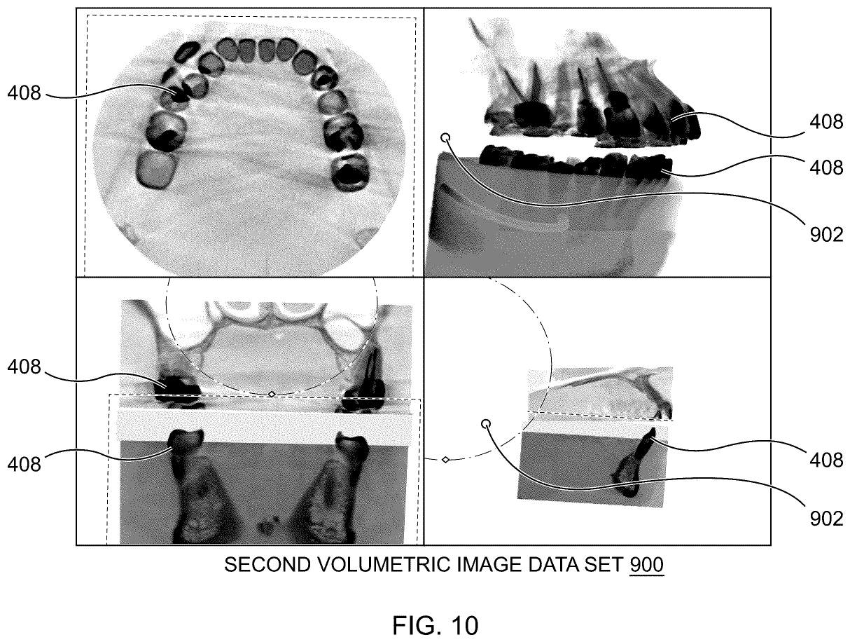

The simulated hinging motion between the modeled upper arch 402 and modeled lower arch 404 is exemplified by the aspect shown in FIG. 10. In FIG. 10, the modeled lower arch 404 has been rotated slightly about the pivot point from the closed-bite position shown in FIG. 9 to an open-bite position wherein the modeled teeth 408 of the modeled upper arch 402 and the modeled lower arch 404 are spaced apart. The relative distance between the modeled rows of teeth of the two modeled arches may be increased or decreased using suitable controls provided by the positioning module 312.

Although a hinging motion is shown in FIG. 10, the reference point 902 need not be a pivot point. Other adjustments may be simulated such as linear translational movement, protrusive or retrusive movement, excursive movement, inferior or superior translation, or rotational movement between the modeled lower arch 404 and the modeled upper arch 402.

While the invention has been described in terms of specific aspects, it is apparent that other forms could be adopted by one skilled in the art. For example, the methods described herein could be performed in a manner which differs from the aspects described herein. The steps of each method could be performed using similar steps or steps producing the same result but which are not necessarily equivalent to the steps described herein. Some steps may also be performed in different order to obtain the same result. Similarly, the apparatuses and systems described herein could differ in appearance and construction from the aspects described herein, the functions of each component of the apparatus could be performed by components of different construction but capable of a similar though not necessarily equivalent function, and appropriate materials could be substituted for those noted. Accordingly, it should be understood that the invention is not limited to the specific aspects described herein. It should also be understood that the phraseology and terminology employed above are for the purpose of disclosing the illustrated aspects, and do not necessarily serve as limitations to the scope of the invention.

What is claimed is:

1. A method comprising:
acquiring a first volumetric image data set representing dentition of a patient, the first volumetric image data set including a modeled upper arch and a modeled lower arch;
segmenting the modeled upper arch and the modeled lower arch in the first volumetric image data set;
for each of the modeled upper arch and the modeled lower arch, masking a one of the modeled upper arch and the modeled lower arch to obtain decoupled volumetric image data including an other one of the modeled upper arch and the modeled lower arch; and,
combining the decoupled volumetric image data including the modeled upper arch and the decoupled volumetric image data including the modeled lower arch into a second volumetric image data set representing dentition of the patient and having the modeled upper arch and the modeled lower arch decoupled from one another.

2. The method of claim 1, further comprising:
modifying the second volumetric image data set to include at least one reference point relative to which one of the modeled upper arch and the modeled lower arch is repositioned relative to the other one of the modeled upper arch and the modeled lower arch.

3. The method of claim 2, wherein the one of the modeled upper arch and the modeled lower arch is selectively repositioned relative to the other one of the modeled upper arch and the modeled lower arch.

4. The method of claim 1, wherein the reference point is a pivot point about which one of the modeled upper arch and the modeled lower arch is rotated for repositioning the one of the one of the modeled upper arch and the modeled lower arch relative to the other one of the modeled upper arch and the modeled lower arch.

5. The method of claim 4, wherein the one of the modeled upper arch and the modeled lower arch is rotatable about the pivot point between an open bite position wherein the modeled upper arch and the modeled lower arch are in contact with one another and a closed bite position wherein the modeled upper arch and the modeled lower arch are spaced apart.

6. The method of claim 1, further comprising:
exporting the second volumetric image data set to a Digital Imaging and Communications in Medicine (DICOM) file format.

7. The method of claim 1, wherein masking further comprises:
providing a horizontal boundary one of above and below an occlusal plane between the modeled upper arch and the modeled lower arch; and,
masking one of the modeled upper arch and the modeled lower arch that extends from the horizontal boundary and away from the other one of the modeled upper arch and the modeled lower arch.

8. The method of claim 7, further comprising:
masking portions of the one of the modeled upper arch and the modeled lower arch that extend beyond the horizontal boundary toward the other one of the modeled upper arch and the modeled lower arch.

9. A system comprising:
a capture module configured to acquire a first volumetric image data set representing dentition of a patient, the first volumetric image data set including a modeled upper arch and a modeled lower arch;
a segmentation module configured to segment the modeled upper arch and the modeled lower arch in the first volumetric image data set;
a masking module configured to, for each of the modeled upper arch and the modeled lower arch, mask a one of the modeled upper arch and the modeled lower arch to obtain decoupled volumetric image data including an other one of the modeled upper arch and the modeled lower arch; and,
a modeling module configured to combine the decoupled volumetric image data including the modeled upper arch and the decoupled volumetric image data including the modeled lower arch into a second volumetric image data set representing dentition of the patient and having the modeled upper arch and the modeled lower arch decoupled from one another.

10. The system of claim 9, further comprising:
a positioning module configured to modify the second volumetric image data set to include at least one reference point relative to which one of the modeled upper arch and the modeled lower arch is repositioned relative to the other one of the modeled upper arch and the modeled lower arch.

11. The system of claim 10, wherein the one of the modeled upper arch and the modeled lower arch is selectively repositioned relative to the other one of the modeled upper arch and the modeled lower arch.

12. The system of claim 9, wherein the reference point is a pivot point about which one of the modeled upper arch and the modeled lower arch is rotatable for repositioning the one of the modeled upper arch and the modeled lower arch relative to the other one of the modeled upper arch and the modeled lower arch.

13. The system of claim 12, wherein the one of the modeled upper arch and the modeled lower arch is rotatable about the pivot point between an open bite position wherein the modeled upper arch and the modeled lower arch are in contact with one another and a closed bite position wherein the modeled upper arch and the modeled lower arch are spaced apart.

14. The system of claim 9, further comprising:
an export module configured to export the second volumetric image data set to a Digital Imaging and Communications in Medicine (DICOM) file format.

15. The system of claim 9, wherein the masking module is further configured to:
provide a horizontal boundary one of above and below an occlusal plane between the modeled upper arch and the modeled lower arch; and,
mask one of the modeled upper arch and the modeled lower arch that extends from the horizontal boundary and away from the other one of the modeled upper arch and the modeled lower arch.

16. The system of claim 15, wherein the masking module is further configured to:
mask portions of the one of the modeled upper arch and the modeled lower arch that extend beyond the horizontal boundary toward the other one of the modeled upper arch and the modeled lower arch.

17. A non-transitory computer-readable medium having instructions stored thereon which, when executed on a processor, perform the steps of:
acquiring a first volumetric image data set representing dentition of a patient, the first volumetric image data set including a modeled upper arch and a modeled lower arch;
segmenting the modeled upper arch and the modeled lower arch in the first volumetric image data set;
for each of the modeled upper arch and the modeled lower arch, masking a one of the modeled upper arch and the modeled lower arch to obtain decoupled volumetric image data including an other one of the modeled upper arch and the modeled lower arch; and,
combining the decoupled volumetric image data including the modeled upper arch and the decoupled volumetric image data including the modeled lower arch into a second volumetric image data set representing dentition of the patient and having the modeled upper arch and the modeled lower arch decoupled from one another.

18. The non-transitory computer-readable medium of claim 17, further comprising instructions for performing the step of:
modifying the second volumetric image data set to include at least one reference point relative to which one of the modeled upper arch and the modeled lower arch is repositioned relative to the other one of the modeled upper arch and the modeled lower arch.

19. The non-transitory computer-readable medium of claim 18, wherein the one of the modeled upper arch and the modeled lower arch is selectively repositioned relative to the other one of the modeled upper arch and the modeled lower arch.

20. The non-transitory computer-readable medium of claim 17, wherein the reference point is a pivot point about which one of the modeled upper arch and the modeled lower arch is rotated for repositioning the one of the one of the modeled upper arch and the modeled lower arch relative to the other one of the modeled upper arch and the modeled lower arch.
